(19) United States
(12) Patent Application Publication
(10) Pub. No.: US 2009/0012428 A1
(43) Pub. Date: Jan. 8, 2009

(54) REUSABLE PIERCING AID AND METHOD FOR CARRYING OUT A PIERCING MOVEMENT BY MEANS OF A REUSABLE PIERCING AID

(76) Inventors: Uwe Kramer, Ilvesheim (DE); Joachim Hoenes, Zwingenberg (DE); Karl Miltner, Frankenthal (DE); Hans List, Hesseneck-Kailbach (DE); Michael Keil, Ludwigshafen (DE)

Correspondence Address: BAKER & DANIELS LLP / ROCHE, 300 NORTH MERIDIAN STREET, SUITE 2700, INDIANAPOLIS, IN, 46204, US

(21) Appl. No.: 12/132,372

(22) Filed: Jun. 3, 2008

Related U.S. Application Data
(63) Continuation of application No. PCT/EP2006/069124, filed on Nov. 30, 2006.

(30) Foreign Application Priority Data
Dec. 5, 2005 (EP) EP2005/026526.3

(51) Int. Cl. A61B 5/151; A61B 17/32

(52) U.S. Cl. 600/583; 606/181; 606/182

(57) ABSTRACT

The invention relates to a reusable piercing aid, containing a lancet, an energy store for transmission of energy stored in the energy store to the lancet in order to carry out a piercing movement, a charging device for charging the energy store, and a control unit, wherein the control unit is designed in such a way that, following the piercing movement, it controls the charging device such that, following the piercing movement, the energy store is automatically charged with energy for the next piercing movement of the lancet.

### RELATED APPLICATIONS

This application is a continuation of and claims priority to PCT/EP06/069124, filed Nov. 30, 2006, which is based on EP 05026526.3, filed on Dec. 5, 2005, the entire disclosures of which is expressly incorporated herein by reference.

### FIELD OF THE INVENTION

The present invention relates to a reusable piercing aid and to a method for carrying out a piercing movement by means of a reusable piercing aid, in particular a method for creating an opening in the skin in part of the body.

A body fluid (blood or interstitial fluid) can then be collected from the opening in the skin.

Samples of body fluids, in particular blood, are taken mainly with the aim of carrying out a subsequent analysis, in order to permit diagnosis of diseases or to monitor the metabolic status of a patient. Such samples are taken by diabetics in particular, for determining the blood sugar concentration. In order to collect only small quantities of blood for diagnostic purposes, for example, sterile, sharp lancets are normally used which, for example, are briefly inserted, by hospital staff or by the patients themselves, into the finger pad or into other parts of the body. In the area of home monitoring in particular, where persons without specialist medical training carry out simple analyses of blood themselves, lancets and associated devices (blood sampling devices, blood lancet devices or, as they are referred to in the following, piercing aids) are sold which allow samples of blood to be taken with the least possible pain and in a reproducible manner.

In the prior art, piercing aids have for some time been known which contain mechanical or electrical drive units for a lancet or a needle, with which the patient or hospital staff can withdraw fluid in a simple manner. An example of an appliance suitable for this purpose is the commercially available “Softclix”, whose operation is described in U.S. Pat. No. 5,318,584. This appliance provides a possibility of setting the depth of insertion of a lancet into the tissue. Thus, the patient is able to select the minimum depth of insertion with which he obtains just the right amount of blood for subsequent analysis, and he can thus minimize the pain caused by puncturing the skin. During a piercing operation using this piercing aid, a compressed spring transmits energy to a lancet in order to carry out a piercing movement.

US 2004/0092996 A1 relates to a further blood sampling system with a lancet driven by a spring. This piercing aid has means for tensioning the spring. In order to drive the lancet, a drive rotor is driven by the spring, and the resulting rotation movement of the drive rotor is converted by a coupling mechanism into the piercing movement of the lancet.

In other piercing aids known in the prior art, the user is for the most part spared the sometimes awkward task of tensioning the lancets and of then triggering the piercing operation. By pressing a button, the patient can activate an electrical drive mechanism, without the need for any further maneuvers and without any force having to be applied by the user. Document WO 02/100460 discloses a blood sampling system in which an electrical drive unit is used to move the lancet. The movement of the lancet is controlled by control units, such that a defined piercing movement can take place.

The liquid sample obtained by creating an opening in the skin by means of a piercing aid is, for example, analyzed by an analysis system that determines the blood sugar content. In the prior art, analysis systems are known in which a blood sample is analyzed electrochemically or photometrically on an analytical test element (e.g. a test element as is described in CA 2,311,496). However, the numerous system components (lancet, piercing aid, test element and analysis appliance) needed for independent blood sugar determination require a lot of space and result in relatively complex handling. There are now also systems with a greater degree of integration and, therefore, simpler handling, in which, for example, the test elements are stored in a magazine in the analysis system and are made available for the measurement. An integrated analysis system can also include a piercing aid, such that, ideally, the creation of an opening in the skin, the collection of a blood sample on a test element and the carrying out of measurements for analysis of the sample on the test element are effected automatically by the analysis system.

WO 02/00101 discloses an analysis system with a large number of needles that can be moved individually out of a housing by a pressing means that is driven electrically or by a spring, so as to perforate the skin. The sample thus obtained is analyzed in the analysis system.

### BACKGROUND AND SUMMARY OF THE INVENTION

In a reusable piercing aid, either standing alone or integrated in an analysis system, an energy store has to be charged before carrying out a piercing movement of the lancet, such that the energy stored therein can be converted at least partially to the kinetic energy of a lancet. A spring provided as energy store in the analysis system, for example, has to be tensioned in order to carry out a piercing movement of a lancet. The manual tensioning of a spring by a user, prior to use of the piercing aid, represents an added operating step that should be avoided. An automatic charging of the energy store (e.g. tensioning of the spring) in the piercing aid or in the analysis system, before the piercing aid is activated, either entails a long waiting time or requires a high level of power, for example of an electrical drive, and therefore a high energy consumption, as a result of which there is more rapid discharging of the batteries present.

The present invention includes a piercing aid and a method for carrying out a piercing movement by means of a reusable piercing aid, where the low energy is needed per measurement cycle, and in particular a piercing aid and a method where the reusable piercing aid can be made ready for use as quickly in order to carry out a piercing movement, e.g. for creating an opening in the body.

According to the invention, a reusable piercing aid includes a lancet, an energy store for transmission of energy stored in the energy store to the lancet in order to carry out a piercing movement, a charging device for charging the energy store, and a control unit, wherein the control unit is designed in such a way that, following the piercing movement, it controls the charging device such that, following the piercing movement, the energy store is automatically charged with energy for the next piercing movement of the lancet.

The invention further relates to a method for carrying out a piercing movement by means of a reusable piercing aid with a lancet contained in the piercing aid, in which the piercing movement is effected by transmission of energy stored in an energy store to the lancet, and, following the piercing movement, the energy store is automatically charged with energy for the next piercing movement of the lancet. The piercing movement preferably serves to create an opening in the skin in a part of the body. The opening in the skin is preferably created in a finger pad, but it can also be created at any other desired part of the body.

A piercing movement is in this connection a movement of the lancet in which the tip of the lancet is moved out through an opening in the housing of the piercing device, or in the housing of an analysis system, to such an extent that it can penetrate sufficiently into the skin of the part of the body that is resting on the opening of the housing. After the piercing movement, the lancet is preferably moved back completely into the housing, in order to avoid accidental piercing.

In connection with the invention, a lancet can be used independently of other components, in particular independently of a test element, or can be used, for example, as a component part of a disposable in the piercing aid. Several functions or functional elements are integrated in an analytical aid (disposable). In particular, the analytical aid can comprise a lancet and a test element (e.g. described in US 2003/0050573 A1 or in US 2002/0052618 A1).

To achieve piercing with the least possible pain, the lancet should experience great acceleration, such that a piercing movement takes place in which the lancet penetrates at high speed into the part of the body. For this purpose, in the method according to the invention, an energy store is provided in which the stored energy can be converted at least partially to kinetic energy of the lancet. The energy store is largely uncharged after a piercing operation. According to the invention, following the piercing movement the energy store is automatically supplied with energy for the next piercing movement. Here, “following” means, at the earliest, immediately after the piercing movement has been carried out (or immediately after the opening has been created in the skin) and, at the latest, before the piercing aid, or an analysis appliance comprising the piercing aid, is moved to a rest state, which lasts until the next use of the piercing aid or of the analysis appliance.

The energy store is supplied with energy for the next piercing movement by a charging device, which provides this energy and which, following the piercing movement, is controlled by a control unit in such a way that it delivers the energy to the energy store at this time. The control unit can, for example, be a processor contained in the piercing aid or in an analysis appliance comprising the piercing aid.

In the method according to the invention, the energy store is charged after an associated piercing operation of the piercing aid (if appropriate after a measurement is carried out in the analysis appliance), and not just shortly before a renewed use of the piercing aid. The piercing aid is therefore immediately ready for use when the piercing aid or the analysis system is set in operation again. Charging of the energy store (e.g. tensioning of a spring) no longer has to take place at this time.

Following a piercing movement, however, there is usually quite a long time interval during which the piercing aid is not needed, with the result that charging of the energy store can be done without any hurry. If, for example, an electric motor is used as the charging device for charging the energy store, a relatively simple, weak motor can be used which runs slightly longer but in doing so consumes little energy. The useful life of the piercing aid (batteries and mechanism) can thus be increased, and the costs of the piercing aid or the analysis appliance can be reduced.

According to another aspect of the invention, manual tensioning of a spring or similar device in order to prepare for a piercing operation is not necessary.

In the method according to the invention, tensioned piercing aids are less sensitive to mechanical influences during the rest phase prior to renewed use. Loose and untensioned mechanisms can move during transport of the appliance and damage or knock out guides. In the method according to the invention, tensioned systems do not have this degree of freedom and therefore have a longer useful life.

The energy store preferably includes at least one energy store selected from the group comprising at least a spring, an electrical energy store and a pressure means. The spring stores energy by tensioning (compression or expansion). The electrical energy store is charged with electrical energy. The pressure means, in the charged state, contains a compressed gas or gas mixture which, when released, can deliver its energy to the lancet for carrying out a piercing movement.

The charging device preferably includes at least one charging device selected from the group comprising a motor coupled to the energy store, a controllable pump for compression of a gas or gas mixture, a pressure reservoir containing a gas or gas mixture and having a controllable valve, an accumulator with a controllable switch, and a battery with a controllable switch. The motor coupled to the energy store can, for example, be an electric motor which can be controlled by a control unit and serves to tension a spring provided as energy store for the next piercing operation. A pressure means provided as energy store can, for example, be supplied with energy for the next piercing operation with the aid of a controllable pump for compression of a gas or gas mixture. For this purpose, however, a pressure reservoir can also be provided that contains a compressed gas or gas mixture, the pressure reservoir delivering the compressed gas or gas mixture to the pressure means as soon as the control unit opens a controllable valve. An electrical energy store can be a capacitor or an accumulator, for example. An electrical energy store can be supplied with electrical energy for the next piercing operation by an accumulator or battery provided as charging device, in which case an electrical connection for the charging operation can be established between energy store and charging device by control of the controllable switch by the control unit.

According to a preferred embodiment of the present invention, the piercing movement is effected by energy stored in a tensioned spring being transmitted to the lancet, the spring being tensioned again following the piercing movement. The spring serves here as energy store, and the “charging” of the energy store is effected by tensioning of the spring. Tensioning in this connection signifies either the compression or the expansion of the spring. The spring used can be, for example, a helical spring, a torsion spring or a leg spring. When a piercing operation is triggered, the spring is guided to an untensioned state. The released force is used to drive the lancet and carry out the piercing movement. The spring is distinguished by a rapid speed of energy release and can provide the piercing aid, within a few milliseconds, with the energy needed for carrying out the piercing operation. The lancet is preferably coupled to the spring via a coupling mechanism such that the energy stored in the tensioned spring can be transmitted largely as kinetic energy to the lancet. The lancet preferably carries out a positively guided piercing movement.

According to another embodiment of the present invention, electrical energy stored in the energy store is transmitted to the lancet largely as kinetic energy for carrying out a piercing movement, and the energy store is charged with electrical energy following the piercing movement. Such an energy store, which stores electrical energy, can be a capacitor or accumulator, for example. In order to carry out a piercing movement, the electrical energy from the energy store is preferably transmitted to the lancet by means of an electromagnet or plunger-type coil.

According to another embodiment of the present invention, the energy store contains a gas or gas mixture, and, following the piercing movement of the lancet, the energy store is charged with energy for the next piercing movement of the lancet by compression of the gas or gas mixture and by delivery of the compressed gas or gas mixture. The energy store can, for example, be a compressed air store which, by release of the compressed air contained in it, transmits its energy to the lancet as kinetic energy.

In the present invention, a motor coupled to the energy store preferably provides the energy for charging the energy store. For example, a spring provided as energy store can be compressed by an electric motor.

For charging the energy store, in particular for tensioning the spring, the motor can be coupled to the spring via a coupling and/or via a gear. The coupling used between motor and energy store can, for example, be a coupling controlled in terms of torque or angle of rotation. The gear used can, for example, be a bevel gear pair. However, it is conceivable to use all coupling types and gear types known in the prior art that permit transmission of energy from the motor to the energy store. According to one embodiment of the present invention, the motor additionally provides energy for a further system function, independent from the energy store, of an analysis system for analyzing a liquid sample. The motor then serves as a combined drive that provides energy both for charging the energy store (e.g. for tensioning a spring) and also for at least one further system function. For example, the further system function can be the transport of a test element magazine and/or of an individual test element in an analysis system. The motor can in this case be used in succession or simultaneously for the different functions. The motor preferably drives a transport system that transports test elements to a sample collection position and to a measurement position. The test element in this case is preferably a test strip that can be evaluated electrochemically or photometrically and that comprises a test field, the test field containing a test chemical that can react with an analyte in a sample.

The sample collection position of a test element in an analysis system is the position in which the test element is ready to receive a sample, for example of blood or interstitial fluid. For example, the test element can have an end with a sample application site protruding from a slit in the housing of an analysis system, such that a user is able to transfer a sample of blood, from a part of the body in which an opening has been created in the skin by the method according to the invention, onto the sample application site.

The measurement position of a test element in an analysis system is the position in which a measurement is carried out in order to analyze a sample on the test element.

A great many methods are known for measuring the concentration of analytes, for example glucose in a blood sample. Such methods usually fall into two categories: optical methods or electrochemical methods.

Optical methods are based on color changes that occur in the course of the detection reaction in the presence of the analytes that are to be determined. The color change occurring on the test element can be detected by reflection photometry. A measurement of transmission is also possible, but this necessitates at least partially transparent test strips.

Electrochemical methods for determination of the concentration of an analyte are based, for example, on amperometry or coulometry.

To perform electrochemical analysis, electrical signals have to be transmitted between the test element and the analysis system. Therefore, a test element introduced into an analysis system has to be electrically contacted in the analysis system with the aid of an electrical connection system.

The optical or electrochemical analysis takes place while the test element is located in the measurement position. In the context of the present invention, the measurement position can be the same position of the test element in the analysis system as the sample collection position, or it can be a position different than this.

According to a preferred embodiment of the present invention, the method according to the invention is performed in an analysis system containing a test element magazine, for storing at least two test elements, and a test element withdrawal device or a transport system for automatically withdrawing a test element from the test element magazine and/or for transporting the test element to the sample collection position and/or measurement position in the analysis system. Various test element magazines are known in the prior art.

DE 198 19 407 discloses, for example, a container for blood sugar meters or other measurement appliances that operate with disposable test strips that can be fed to a sensor for measurement, the container being composed of two parts, in the first of which the test strips are stored, and in the second of which the used test strips are collected. The test strips can be arranged in series such that they form a tape, which can be spooled similarly to a music cassette tape. They can instead also be arranged such that they form a round disk on which they are arranged at a defined distance from one another in the area of the disk circumference, such that, by rotation of the disk, a new test field arrives at the corresponding measurement position. A further possibility is that the test strips form a stack, which is processed individually by a motor-driven mechanism and brings the test strips one after another to the corresponding sample collection position and/or measurement position and, after completion of the measurement, to a collection compartment.

DE 198 54 316 A1 describes a drum-shaped test element magazine in which test elements are held in separate chambers that are impervious to water vapor. Each of the chambers has at least two openings that lie opposite each other and are each closed by a sealing film. To remove the test elements, a ram driven by a motor pushes a test element out of its chamber. The ram breaks through the sealing film on one side of the chamber and then presses against the test element which, because of this pressure of the ram, breaks through the sealing film on the opposite side, such that the test element can be pushed out of the chamber and into the sample collection position and/or measurement position.

According to a preferred embodiment of the present invention, the reusable piercing aid according to the invention is a component part of an analysis system in which an analysis of a liquid sample on a test element can be carried out.

According to a preferred embodiment of the present invention, a large number of test elements are provided in a tape-like test element magazine. An analysis of a liquid sample then preferably takes place as follows:
a) A body part of the user is applied to a housing opening of a piercing aid, or of an analysis system containing a piercing aid, since in the method according to the invention the lancet of the piercing aid preferably emerges from a housing opening of the piercing aid onto which the body part is to be applied in order to create an opening in the skin.
b) Triggering of the piercing operation, such that the energy of the energy store (e.g. of the tensioned spring) is transmitted at least partially to the lancet for carrying out the piercing movement.
c) If appropriate, when carrying out the piercing movement, the lancet additionally passes through a test element arranged behind the housing opening, e.g. as part of a test element tape. For this purpose, a correspondingly positioned puncture opening can be provided in the test element, such that there is no resistance to the piercing movement of the lancet.
d) The lancet carries out a piercing movement, and the tip of the lancet emerges from the housing opening, creates an opening in the skin in the body part of the user and is drawn back into the piercing aid again through the housing opening and, if appropriate, the puncture opening of the test element.
e) The tape with the test elements is, if appropriate, moved on further until the test element to be used is located in the sample collection position.
f) The liquid sample from the body part of the user is applied to the test element.
g) A measurement is then carried out on the sample (in the same position, or the tape with test elements is moved on further to another measurement position).
h) The energy store is then charged (in particular the spring is tensioned), such that the piercing aid or analysis system is ready for further implementation of the method according to the invention for carrying out a piercing movement. If appropriate, the tape with test elements is also moved on farther, such that it is likewise ready for the next piercing operation or analysis procedure.
i) This is then usually followed by a rest phase until the piercing aid/analysis system is put to use once more.

Steps a) to h) can take place in succession in the stated sequence or in another sequence, or at least some of them can take place at the same time. According to the invention, step h) always takes place after step d) and before step i).

The charging of the energy store in step h) and preferably also the movement of the test elements tape in steps e), g) and h) are controlled by a control unit. To charge the energy store, the control unit controls a charging device which is provided for this purpose and which then supplies the energy store with energy for the next piercing operation.

According to another embodiment of the present invention, for each piercing operation by the method according to the invention, an individual test element or disposable is used which the user inserts manually into the analysis system following a piercing operation and if appropriate a measurement operation or, in the case of a test element, directly before the next piercing operation.

The invention further relates to the use of a piercing aid for carrying out a piercing movement (in particular for creating an opening in the skin in a part of the body) by means of a reusable piercing aid with a lancet contained in the piercing aid, the piercing movement being effected by transmission of energy stored in an energy store to the lancet, characterized in that, following the piercing movement, the energy store is automatically charged with energy for the next piercing movement of the lancet.

### BRIEF DESCRIPTION OF THE DRAWINGS

The invention is explained in more detail below with reference to the drawing, in which:

FIG. 1 shows a piercing aid for carrying out a piercing movement by the method according to the invention.

### DETAILED DESCRIPTION OF EMBODIMENTS OF THE INVENTION

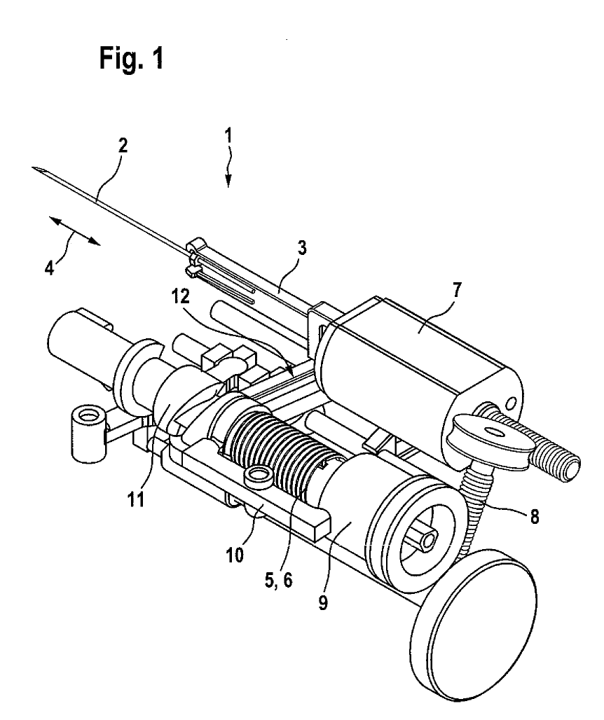

The piercing aid 1 can be a component part of an analysis system (not shown) or can stand alone. It comprises a lancet 2, which is held by a lancet body 3. The lancet 2 and the lancet body 3 can together be moved linearly in the longitudinal direction 4, in order to carry out a piercing movement. In the embodiment shown in FIG. 1, the energy store 5 is a spring 6, which can provide the energy for the piercing movement.

This energy is stored in the energy store 5 by tensioning of the spring 6. The spring 6 is tensioned by a motor 7 which is provided as charging device and which can rotate the tensioning rotor 9 via a gear 8. The motor 7 can be controlled by a control unit (not shown). The spring 6 is tensioned by the rotation movement of the tensioning rotor 9, until it has stored sufficient energy for the piercing movement.

To trigger the piercing movement, the tensioning rotor 9 is rotated by the motor 7 by a small predefined angle in the tensioning direction until a cam (not shown) on the tensioning rotor 9 actuates the toggle lever 10, which presses a trigger element (not shown) of this drive rotor 11. The energy of the spring 6 is then transmitted to the drive rotor 11, as a result of which the lancet body 3 and the lancet 2 are driven by means of a control cam and a lever 12 in order to carry out a piercing movement. The energy stored in the energy store 5 (spring 6) is converted at least partially to the kinetic energy of the lancet 2 in the piercing operation. According to the invention, following the piercing movement, the energy store 5 is automatically charged for the next piercing movement of the lancet 2. This is achieved by control of the motor 7 by the control unit (not shown) and by renewed tensioning of the spring 6 with the aid of the motor 7 and of the gear 8.

1. A reusable piercing aid, including:
a lancet,
an energy store for transmission of energy stored in the energy store to the lancet in order to carry out a piercing movement,
a charging device for charging the energy store, and
a control unit configured to control the charging device such that, following the piercing movement, the energy store is automatically charged with energy for the next piercing movement of the lancet.

2. The piercing aid of claim 1, wherein the energy store includes one of a spring, an electrical energy store and a pressure energy store.

3. A method for carrying out a piercing movement by means of a reusable piercing aid with a lancet contained in the piercing aid, the piercing movement being effected by transmission of energy stored in an energy store to the lancet, the method including the step of:
controlling a charging device in such a way that, following the piercing movement, the charging device automatically charges the energy store with energy for the next piercing movement of the lancet.

4. The method of claim 3, wherein the piercing movement is effected by energy stored in a tensioned spring being transmitted to the lancet, the spring being tensioned again following the piercing movement.

5. The method of claim 3, wherein electrical energy stored in the energy store is transmitted to the lancet at least partially as kinetic energy for carrying out a piercing movement, and the energy store is charged with electrical energy following the piercing movement.

6. The method of claim 3, wherein the energy store contains a gas, and, following the piercing movement of the lancet, the energy store is charged with energy for the next piercing movement of the lancet by compression of the gas or gas mixture.

7. The method of claim 3, wherein the charging device includes a motor coupled to the energy store to provide the energy for charging the energy store.

8. The method of claim 7, wherein the motor additionally provides energy for a further system function, independent from the energy store, of an analysis system for analyzing a liquid sample.

9. The method of claim 7, wherein the motor drives a transport system that transports test elements to a sample collection position and to a measurement position.

10. The method of claim 3, wherein, when carrying out the piercing movement, the lancet emerges from a housing opening of the piercing aid onto which a body part is to be applied in order to create an opening in skin.

11. The method of claim 3, wherein, when carrying out the piercing movement, the lancet passes through a test element.

12. A method of operating a piercing aid with a lancet contained in the piercing aid, including the steps of:
transmitting energy stored in an energy store to cause the lancet to carry out a piercing movement, and
automatically charging the energy store following the piercing movement with energy for the next piercing movement of the lancet.

13. The piercing aid of claim 2, wherein the charging device includes one of a motor coupled to the energy store, a controllable pump for compression of a gas, a pressure reservoir containing a gas and having a controllable valve, an accumulator with a controllable switch, and a battery with a controllable switch.

14. The piercing aid of claim 1, wherein the energy store is a coil spring.

15. The piercing aid of claim 1, further including a gear coupled between the charging device and the energy store.

16. The piercing aid of claim 1, further including a drive rotor coupled to the energy store, the drive rotor being configured to drive a lever coupled to the lancet to cause the lancet to carry out the piercing movement.

17. The method of claim 12, wherein the transmitting step includes the step of causing a drive rotor to rotate, thereby moving a lever that causes the lancet to carry out the piercing movement.

18. The method of claim 12, wherein the energy store is a spring.

19. The method of claim 13, wherein the charging step includes the step of controlling a motor to impart energy to the spring.

20. The method of claim 19, wherein the controlling step includes the step of causing rotation of a gear coupled to the motor and the spring.
